(19) United States
(12) Patent Application Publication
(10) Pub. No.: US 2020/0388358 A1
(43) Pub. Date: Dec. 10, 2020

(54) Machine Learning Method for Generating Labels for Fuzzy Outcomes

(71) Applicant: GOOGLE LLC, Mountain View, CA (US)

(72) Inventors: Kai CHEN, San Bruno, CA (US); Kun ZHANG, Mountain View, CA (US); Jacob MARCUS, Mountain View, CA (US); Eyal OREN, Los Gatos, CA (US); Hector YEE, Mountain View, CA (US); Michaela HARDT, Mountain View, CA (US); James WILSON, Littleton, CA (US); Alvin RAJKOMAR, Mountain View, CA (US); Jian LU, Sunnyvale, CA (US)

(21) Appl. No.: 16/636,989

(22) PCT Filed: Sep. 29, 2017
(86) PCT No.: PCT/US2017/054215

§ 371 Date: Feb. 6, 2020

Related U.S. Application Data
(60) Provisional application No. 62/552,011, filed on Aug. 30, 2017.

(51) Int. Cl. G16H 10/60; G06N 20/00

(52) U.S. Cl. CPC G16H 10/60; G06N 20/00

(57) ABSTRACT

A machine learning method is described for generating labels for members of a training set where the labels are not directly available in the training set data. In a first stage of the method an iterative process is used to gradually build up a list of features (“partition features” herein) which are conceptually related to the class label using a human-in-the loop (expert). In a second part of the process we generate labels for the members of the training set, build up a boosting model using the labeling to come up with additional partition features, score the labeling of the training set members from the boosting model, and then with the human-in-the-loop evaluate a labels assigned to a small subset of the members depending on their score. The labels assigned to some or all of those members in the subset may be flipped depending on the evaluation. The final outcome of the process is an interpretable model that explains how the labels were generated and a labeled set of training data.

### PRIORITY

This application claims priority to U.S. Provisional Application Ser. No. 62/552,011 filed Aug. 30, 2017.

### BACKGROUND

This disclosure relates to the field of machine learning and more particularly to a way of generating labels for each of the members of a set of training data where the labels are not available in the training data per se. The labels are conceptually associated with some particular characteristic or property of the samples in the training data (a term referred to herein as “outcomes”).

Machine learning models, for example neural network models used in the health sciences to make predictions or establish a predictive test, typically are generated from collections of electronic health records. Some labels are present in the training set used to generate the models and are considered “hard”, for example, in-patient mortality (the patient did or not die in the hospital), transfer to intensive care unit (ICU), i.e., the patient either was or was not transferred to the ICU while they were admitted to a hospital.

On the other hand, in unharmonized data, such as an electronic health record, some concepts that are semantically well defined are difficult to extract or may not be labeled in the training data. In this document the term “unharmonized” means that common terms are named in a way specific to a particular organization, and not uniformly across different organizations. For example, “acetominophen 500 mg” might be known in one particular hospital as “medication 001” and thus the same thing is referred to differently in two different organizations and thus the terms are not harmonized. As another example, whether a patient has received dialysis is conceptually clear at a high level, but in the data there are many different types of dialysis (intermittent hemodialysis, pure ultra-filtration, continuous veno-venous hemodiafiltration) so the underlying data may require a significant number of rules to comprehensively capture this “fuzzy” topic of interest. Additionally, some labels are not explicitly available in the training set, yet there is a need to assign a label to a member of the training set, e.g., to indicate that the member has some particular characteristic.

Adding the labels manually would be time consuming. It would also be subject to human error. Furthermore, since there may be some subjectivity in assigning the labels, the results may be inconsistent, particularly if different health records are labelled by different individuals.

Accordingly, there is a need in the art for predictive models which account for this “fuzzy label” situation. This document represents a scalable solution to this problem and describes a method for generating labels for all the members of the training data. Moreover, we describe the generation of interpretable, coherent and understandable models which are used to generate the labels in the training data. Additionally, the present disclosure allows for construction of additional predictive models, such as clinical decision support models, from the labeled training data.

### SUMMARY

In one aspect, a computer-implemented method is disclosed of generating a class label for members of a set of training data. The method is performed by certain processing instructions which are implemented by the processor of a computer. The training data for each member in the set includes a multitude of features. In the context of an electronic heath records, the members could be, for example, the electronic health records for individual patients, the training data could be the time sequence data in an electronic health record for the patient, and the features could be things such as vital signs, medications, diagnoses, hospital admissions, words in clinical notes, etc. in the electronic health records. The class label generated by the method is a label which is “fuzzy”, that is, not explicitly available in the training data.

The method includes a first stage of refinement of features related to the class label using input from a human-in-the loop or operator, and includes steps a)-d). In step a) an initial list of partition features which are conceptually related to the class label is received from an operator or human-in-the-loop (i.e., subject matter expert). These initial partition features can be thought of as hints to bootstrap the process. They generally have high precision (i.e., are strongly correlated with the desired label), but low recall (a low proportion of the examples in the training set have the partition feature). The method includes a step b) of using the partition features to label the training data and generate additional partition features related to the class label which are not in the initial list of partition features. Basically, a machine (computer) uses the partition features to generate labels for the training data, e.g., using a decision list or logic defined by the operator. In one embodiment, the machine builds a boosting model to generate or propose additional partition features. The method includes a step c) of adding selected ones of the additional partition features to the initial list of partition features from input by the operator. In essence, the method uses a human-in-the loop to inspect the proposed additional partition features and the “good” ones (based on expert evaluation are added to the partition feature list. The selection could be based on, for example, whether the additional partition features are causally related to the class label.

The method includes step d) of repeating steps b) and c) one or more times to result in a final list of partition features. Basically, in the processing instructions we iterate steps b) and c) several times to generate a final list of partition features.

The computer-implemented process continues to a second stage of label refinement using input from human evaluation of labels. The second stage includes step e) of using the final list of partition features from step d) to label the training data; step f) building a further boosting model using the labels generated in step e); step g) scoring the training examples with the further boosting model of step f), and step h) generating labels for a subset of the members of the training examples based on the scoring of step g) with input from the operator. For example, we may use a known scoring metric such as F1, and select a threshold based on this metric, and for members of the training set which are near the threshold we use a human operator to assign labels to these members, or equivalently inspect the labels that were generated in step e) and either confirm them or flip them based on the human evaluation.

The result of the process is an interpretable model that explains how we generate the fuzzy labels, i.e., the further boosting model from step f), and the labeled training set from steps e) and step h). The labeled training set can then be used as input for generation of other models, such as predictive models for predicting future clinical events for new input electronic health records.

In one embodiment, at least some of the features of the training data are words contained in the health records, and at least some of the partition features are determinations of whether one or more words are present in the health records. In another embodiment, at least some of the features in the training data are measurements in the health records (e.g., vital signs, blood urea nitrogen, blood pressure, etc.), and at least some of the partition features are determinations of whether one or more measurements are present in the health records, for example BUN>=52 mg/dL or that measurement in a given time period.

Additionally, after the human labeling input of step h), we can proceed to build models on the labelled data set, and execute additional “active learning” steps at the end of the procedure to further refine the labels. Thus, in one embodiment we repeat steps f), g) and h) one or more times to further refine the labels, where with each iteration the input for step f) is the labeled training set from the previous iteration.

In another aspect, a computer-implemented method is disclosed of generating a list of features for use in assigning a class label to members of a set of training data. The training data for each member in the set is in the form of a multitude of features. The method is executed in a computer processor by software instructions and includes the steps of: a) receiving an initial list of partition features from an operator which are conceptually related to the class label; b) using the initial list of partition features to label the training data and identify additional partition features related to the class label which are not in the initial list of partition features; c) adding selected ones of the additional partition features to the initial list of partition features from input by an operator to result in an updated list of partition features; and d) repeating steps b) and c) one or more times using the updated list of partition features as the input in step b) to result in a final list of partition features.

In still another aspect, a computer-implemented method is provided for generating a class label for members of a set of training data. The training data for each member in the set is in the form of a multitude of features. The method is implemented in software instructions in a computer processor and includes the steps of: (a) using a first boosting model with input from a human-in-the-loop (operator) to gradually build up a list of partition features; (b) labeling the members of the set of training data with the list of partition features; (c) building a further boosting model from the labeled members of the set of training data and generating additional partition features; (d) scoring the labeling of the members of the set of training data and determining a threshold; (e) identifying a subset of members of the set of training data near the threshold; and (f) assigning labels to the subset of members with input from the human-in-the-loop (operator).

As noted above, it is possible to do further active learning to refine the labels using the human-in-the-loop; hence more models may be built from the labeled training set and we can repeat or iterate the operator assignment of labels. For example we may repeat steps (c), (d), (e) and (f) at least one time and thereby further refine the labels. We may repeat this process several times, each time using as input for step c) the labeled training data from the previous iteration.

The term “boosting model” is here used to mean a supervised machine learning model that learns from labeled training data in which a plurality of iteratively learned weak classifiers are combined to produce a strong classifier. Many methods of generating boosting models are known.

It will be noted that in the broadest sense, the methods of this disclosure can be used for “features” in training data where the term “features” is used in its traditional sense is machine learning as individual atomic elements in the training data which are used to build classifiers, for example individual words in the notes of a medical record, laboratory test results. In the following description we describe features in the form of logical operations which offer more complex ways of determining whether particular elements are present in the training data, taking into account time information associated with the elements. More generally, the methodology may make use of a test (or query) in the form of a function applicable to any member of the training data to detect the presence of one or more of the features in that member of the training data.

Accordingly, in one further aspect of this disclosure a computer-implemented method of generating a respective class label for each of a plurality of members of a set of training data is described. The training data for each member in the set comprises a multitude of features. The method comprising the steps of executing the following instructions in a processor for the computer:

a) receiving an initial list of tests from an operator which are conceptually related to the class label, each test being a function applicable to any member of the training data to detect one or more of the features in that member of the training data;

b) using the tests to label the training data and identify additional tests related to the class label which are not in the initial list of tests;

c) adding selected ones of the additional tests to the initial list of tests based on data from input by the operator;

d) repeating steps b) and c) one or more times to result in a final list of tests;

e) using the final list of tests from step d) to label the training data;

f) building a boosting model using the labels generated in step e);

g) scoring the training examples with the boosting model built in step f) and

h) generating respective labels for a subset of the members of the training examples based on the scoring of step g) with input from the operator.

In one embodiment, in step b) the additional tests are generated using a boosting model.

In one embodiment step f) comprises the steps of initializing the further boosting model with the final list of tests and iteratively generating additional tests building a new boosting model in each iteration. In one embodiment, the iterations of generating additional tests include receiving operator input to deselect some of the generated additional tests.

In one embodiment the scoring step g) comprises determining a threshold related to the score, and identifying members of the training data for which the score differs from the threshold by an amount within a pre-defined range, and wherein step h) comprises the step of generating labels for the identified members of the set of training data.

Once the labels have been assigned per steps e) and h) in one embodiment the method includes a further step of building a predictive model from the set of samples with the labels assigned per steps e) and h).

In one embodiment the members of the set of training data comprises a set of respective electronic health records. Other types of training data could be used in the method besides electronic health records, as the method is generally applicable to assigning fuzzy labels in other situations. In one embodiment, at least some features of the training data are words contained in the health records, and at least some of the tests are determinations of whether one of more corresponding predetermined words are present in the health records, or determinations of whether one or measurements are present.

In one embodiment at least some features of the training data are associated with real values and a time component and are in a tuple format of the type {X, xᵢ, tᵢ} where X is a name of feature, xᵢ is a real value of the feature and tᵢ is a time component for the real value xᵢ; and the tests comprise predicates defined as binary functions operating on sequences of the tuples or logical operations on the sequences of the tuples.

### BRIEF DESCRIPTION OF THE DRAWINGS

FIG. 1 is a flow-chart showing one embodiment of the method of this disclosure.

FIG. 2 is a detailed flow chart showing the step of building the final boosting model (22) of FIG. 1.

FIG. 3 is an illustration of the use of the procedure of FIG. 1 for a training set in the form of electronic health records.

FIG. 4 is a probability histogram as a result of the scoring step of FIG. 1.

### DETAILED DESCRIPTION

This document discloses methods for generating predictive models which account for this “fuzzy label” situation, where training labels are not explicitly available. This document represents a scalable solution to this problem and describes a method for generating labels for a subset or even all the members of the training data. Moreover, we describe the generation of interpretable, coherent and understandable models which are used to generate the labels in the training data. Additionally, the present disclosure allows for construction of additional predictive models, such as clinical decision support models, from the labeled training data. The methods thus have several technical advantages. In addition, in order to generate useful predictive models from electronic health records which have wide applicability there is a need for establishing labels for the training data and without the benefits of the methods this disclosure, such predictive models would be difficult, costly or time consuming to produce or use.

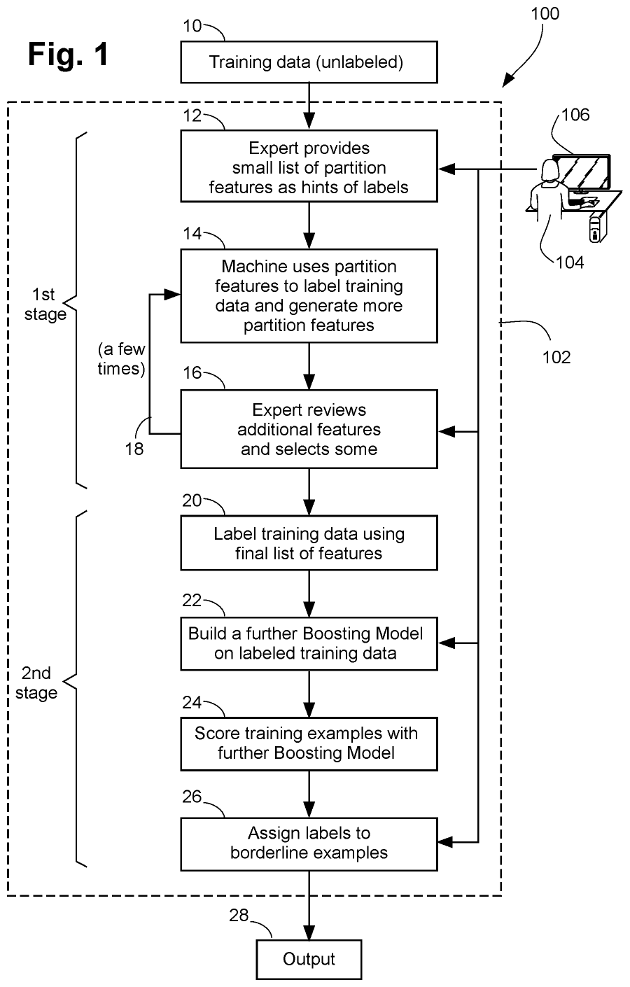

Referring now to FIG. 1, this document describes a computer-implemented method 100 of generating a class label for members of a set of training data 10. The method includes a process 102 executing on a computer which generates an interpretable model that generates class labels for the training data. The output 28 of the process 102 is the interpretable model and the set of training data which is now labeled in accordance with the model.

The training data for each member in the set 10 includes a multitude of features. In the context of an electronic heath records, the members of the training data could be, for example, the electronic health records for individual patients. The training data could be the time sequence data in electronic health records, and the features could be things such as vital signs, medications, diagnoses, hospital admissions, words in clinical notes, etc. found in the electronic health records. We describe later in this document features in the form of “predicates” which are binary functions operating on training data in a tuple format of {feature: real value; time value} of specific features such as laboratory values, vital signs, words in clinical notes, etc. The class label generated by the method 100 is a label which is “fuzzy”, that is, not explicitly available in the training data, hence the training data 10 is initially unlabeled in this regard.

Still referring to FIG. 1, the method indicated by flow chart 102 is performed by software instructions which are executed in a computer. The method 102 includes a first stage of refinement of features related to the class label using input from a human-in-the loop or operator, and includes steps 12, 14, 16 and loop 18. In step 12 an initial list of partition features which are conceptually related to the class label is received from an operator (expert) 104. The operator 104 uses a workstation 106 to specify a small list of features, say one, two, or five or so, that are related to the label. The operator is a domain expert such a medical doctor, and this initial list of features are based on his or her own domain knowledge. These initial partition features can be thought of as “hints” to bootstrap the process. They generally have high precision, but low recall. For example, to label “acute kidney injury” in patients, one initial feature can be “did dialysis occur in the patient history”.

The method includes a step 14 of using the partition features to label the training data and generate additional partition features related to the class label which are not in the initial list of partition features. Basically, a machine (computer) uses the partition features to generate labels for the training data 10. A class label may be assigned based on the partition features using an OR operator, that is if any one of the initial partition features are present in the patient record, it is labelled as a positive example, otherwise it is a negative example. The labeling logic can be more complex than simply using the OR operator, for example, feature 1 OR feature 2 where feature 2 is (feature 2a AND feature 2b). In the context of features taking the form of “predicates” described below, the labeling logic could be two predicates one of which is a composite of two others ANDed together. Since a feature takes a form of “predicate”, it can be very expressive, for example, whether “dialysis” exists within the last week, or a lab test value exceeds a certain threshold. The logic for generating the labels is typically assigned or generated from the human operator.

In one embodiment, the machine builds a boosting model to generate or propose additional partition features, which can be done by constraining the model to not use the initial list of features. For example, the initial feature “did dialysis occur in the patient history” may lead to the following new suggestions: 1) “did hemodialysis occur in patient history”, 2) “did patient have a BUN (blood urea nitrogen) lab test value>=52 mg/dL in the past week”. These additional features are highly correlated with the “acute kidney injury” fuzzy label. The additional partition features could be proposed based on the weighted information gain of randomly selected features, with regard to the labels generated by the partition features.

At step 16, the human operator 104 may select or edit the suggested features, and add them to the initial list of partition features. In essence, the method uses a “human in the loop” 104 to inspect the proposed additional partition features and the “good” ones (based on expert evaluation) are added to the partition feature list, possibly with some edits, for example the lab test result threshold. The selection could be based on, for example, whether the additional partition features are causally related to the class label. This aspect injects expert knowledge into the boosting model and aids in generating an interpretable model that explains in a human understandable manner how the fuzzy labels are assigned to the training data.

The method includes loop step 18 of repeating steps b) and c) one or more times to result in a final list of partition features. Basically, we iterate in software instructions steps 14 and 16 several times to generate a final list of partition features, using as input at each iteration the total list of features resulting at the completion of step 16. This iterative process of steps 14, 16 and loop 18 gradually builds up a boosting model and results in a final list of partition features that are satisfactory to the human operator

The process continues to a second stage of label refinement using input from human evaluation of a subset of the training labels. The second stage includes step 20 of using the final list of partition features from step d) to label the training data. Basically we use the model resulting from steps 14, 16 and 18 repeated many times to generate labels for the training data.

A class label may be assigned based on the final list of partition features by using an OR operator, that is if any one of the partition features are present in the training data for a particular sample (patient data) it is labeled as positive, otherwise it is labelled as negative. The labeling logic can be more complex than simply using the OR operator, for example, feature 1 OR feature 2 where feature 2 is (feature 2a AND feature 2b). In the context of features taking the form of “predicates” described below, the labeling logic could be two predicates one of which is a composite of two others ANDed together, as in the previous example. Since a feature takes a form of “predicate” in the illustrated embodiment, it can be very expressive, for example, whether “dialysis” exists within the last week, or a lab test value exceeds a certain threshold.

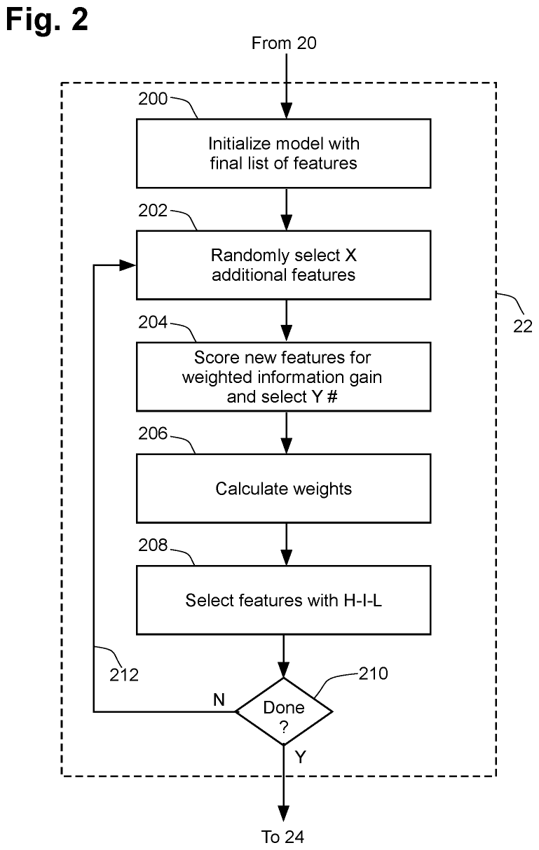

The procedure then proceeds to step 22 in which we build a further boosting model using the labels generated in step 20. Step 22 is shown in more detail in FIG. 2 and will be described below. Basically, in step 22 we initialize the model with the final list of features developed from steps 14, 16 and 18 and then we allow the model to use those features, and ask it to arrive at still additional features when necessary. We optionally use a “human in the loop” 104 to deselect some of the undesirable additional features, e.g., those not causally related to the class label.

At step 24, we score all the training examples with the boosting model of step 22. At step 26 we sample a subset of the examples where their scores in step 24 indicate that the model is not certain about their label, i.e. the model is indecisive about those examples, and give those examples to human experts for further evaluation. For example, we may use a known scoring metric such as F1 and select a threshold based on this metric, and for members of the training set which are near the threshold we use a human operator to assign labels to these members, or, equivalently, inspect the class labels assigned by the machine and either confirm them or flip them. This subset of examples should be much smaller than the entire training data set, hence we save a large amount of expensive and time-consuming human labeling work by using the process 102.

The output 28 of the process 102 is an interpretable model that explains how we generate the fuzzy labels, i.e., the boosting model from step 22, and the labeled training set from steps 20 and step 26. The labeled training set can then be used as input to train other machine learning models, such as predictive models for predicting future clinical events for other patients based on their electronic health records.

Additionally, as noted above, it is possible to do further “active learning” to refine the labels using the human-in-the-loop; hence more models may be built from the labeled training set and we can repeat or iterate the operator assignment of labels in the second stage of the procedure.

An example will now be provided for steps 14 and 16 and 18 and explain how the boosting model is initially constrained to use the initial partition features. More concretely, the partition features are used to select what examples are positive and negative for the boosting model. Then, they are excluded from the boosting model (otherwise the boosting model will just use the partition features itself and no new features will be obtained) in a subsequent iteration of loop 14 and 16. Suppose, in this example, the fuzzy label is “acute kidney injury.”

1. In the first iteration, at step 14 the expert searches for all patients that have ‘dialysis’ in the record (this is the small list of one partition feature provided at step 12). This is done using an initial partition feature (predicate) encoding the query ‘does dialysis exist in the record’.
2. All records that have “dialysis” are considered positive, otherwise not. Boosting is run with these labels, but importantly, excluding the partition predicate ‘does dialysis exist in the record.’
3. Boosting then suggests new predicates like ‘does hemodialysis occur’ or ‘was BUN (blood urea nitrogen) measured.’ As explained above, these partition predicates could be generated by weighted information gain of randomly selected predicates, a procedure described in step 204 of FIG. 2.
4. At step 16 the expert selects ‘hemodialysis’ and ‘BUN’. Now, in the second iteration of loop 18, all patients with dialysis OR hemodialysis OR BUN are considered positive and boosting is run with those labels, excluding the partition predicates ‘dialysis’ and ‘hemodialysis’ and ‘BUN’. In the second iteration of loop 18, at step 14 new partition predicates are proposed (again, using weighted information gain) and at step 16 the expert review and selects some additional partition predicates.
5. Repeat procedure (loop 18) a few more times or until the expert is satisfied with the partition predicates.

FIG. 2 shows one embodiment of how the further boosting model in step 22 is generated. The procedure of step 22 shown in FIG. 2 could also be used to the iterations of steps 14, 16 and 18 of the first stage of the process.

At step 200, we initialize the further boosting model with the final list of features resulting from step 20 of FIG. 1. At step 202, a number (e.g., 5,000) of additional features are selected at random. At step 204, the new randomly selected features are scored for weighted information gain relative to the class label associated with a prediction of the boosting model (e.g., the fuzzy label at issue here), and we select some small number Y of them, where Y is say ten or twenty. The weights for each sample come from computing the probability p of the sample given the current boosting model. The importance q is then q=|label−prediction|. This means that samples that the boosting model makes errors on are more important in the current boosting round. Using the importance q and the label of the samples, one can then compute the weighted information gain of the candidate features with respect to the label and the current boosting model. Alternatively, one can select features randomly and then perform a gradient step with L1 regularization. Another method is to sample groups of features and evaluate for information gain, in accordance with the methods described in https://en.wikipedia.org/wiki/Information_gain_in_decision_trees or use techniques described in the paper of Trivedi et al., An Interactive Tool for Natural Language Processing on Clinical Text, arXiv:1707.01890 [cs.HC] (July 2017).

At step 206 we calculate weights for the selected features with the highest weighted information gain. In this step we then preform a gradient fit to compute weights for all the selected features. We use gradient descent with log loss and L1 regularization to compute the new weights for all previous and newly added features. We use the FOBOS algorithm to perform the fit, see the paper of Duchi and Singer, Efficient Online and Batch Learning Using Forward Backward Splitting, J. Mach. Learn. Res. (2009).

At step 208 we then select, or, equivalently, remove or deselect features in response to operator input, using a human-in-the-loop, such as operator 104 of FIG. 1. In particular, an expert such as a physician 104 operating a computer 106 views the randomly selected features with the highest information gain and then removes those that are deemed not trustworthy or causally unrelated to the prediction task of the model. For example, if one of the features was “number_of_breakfasts” and the prediction task is inpatient mortality, the operator may choose to deselect that feature because it is not causally connected to whether the patient is at risk of inpatient mortality.

At step 210, a check is performed on whether the process of selection of additional features is complete. Normally, the No branch 212 is entered for say ten or twenty iterations, during which time the boosting model is gradually built up consisting of the final list of partition features generated from steps 14, 16 and 18 plus the additional features generated from steps 202, 204, 206 and 208. When a sufficient number of iterations have been completed the process proceeds to step 24 and 26 of FIG. 1 described above, namely scoring the training set using the final boosting model and human-in-the-loop assignment of labels to borderline members of the training set.

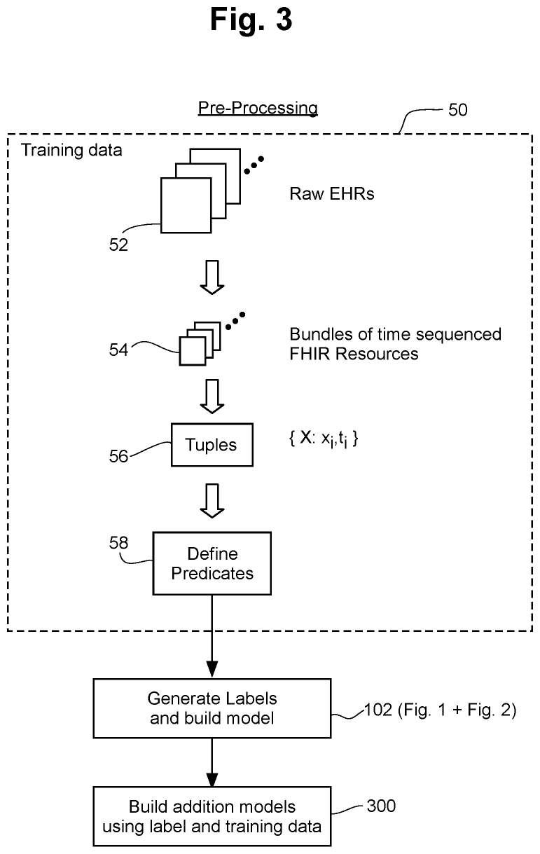

FIG. 3 is an illustration of a procedure for generation of fuzzy labels for training data in the form of electronic health records. In particular, the procedure includes a pre-processing step 50, the procedure 102 of FIGS. 1 and 2 of generating the labels for the training data and construction of an interpretable model that explains how the training data were labeled, and a step 300 of building additional models using the labelled training data.

In the pre-processing step 50, we start with an input dataset 52 of raw electronic health records. In one possible example, this data set could be the MIMIC-III dataset which contains patient de-identified health record data on critical care patients at Beth Israel Deaconess Medical Center in Boston, Mass. between 2002 and 2012. The data set is described in A. E. Johnson et al., MIMIC-III, a freely accessible critical care database, J. Sci. Data, 2016. Of course, other patient de-identified, electronic heath record data sets could be used. It is possible that the dataset 52 could consist of electronic health records acquired from multiple institutions which use different underlying data formats for storing electronic health records, in which case there is an optional step 54 of converting them into a standardized format, such as the Fast Health Interoperability Resources (FHIR) format, see Mandel J C, et al., SMART on FHIR: a standards-based, interoperable apps platform for electronic health records. J Am Med Inform Assoc. 2016; 23(5):899-908, in which case the electronic health records are converted into bundles of FHIR “resources” and ordered, per patient, into a time sequence or chronological order. Further details on step 54 are described in the U.S. provisional patent application Ser. No. 62/538,112 filed Jul. 28, 2017, the content of which is incorporated by reference herein. For the aggregated patient de-identified electronic health records used to create the models, our system includes a sandboxing infrastructure that keeps each EHR dataset separated from each other, in accordance with regulation, data license and/or data use agreements. The data in each sandbox is encrypted; all data access is controlled on an individual level, logged, and audited.

The data in the dataset 52 contains a multitude of features, potentially hundreds of thousands or more. In the example of electronic health records, the features could be specific words or phrases in unstructured clinical notes (text) created by a physician or nurse. The features could be specific laboratory values, vital signs, diagnosis, medical encounters, medications prescribed, symptoms, and so on. Each feature is associated with real values and a time component. At step 56, we format the data in a tuple format of the type {X, xᵢ, tᵢ} where X is the name of feature, xᵢ is a real value of the feature (e.g., the word or phrase, the medication, the symptom, etc.) and tᵢ is a time component for the real value xᵢ. The time component could be an index (e.g., an index indicating the place of the real value in a sequence of events over time), or the time elapsed since the real value occurred and the time when the model is generated or makes a prediction. The generation of the tuples at step 56 is performed for every electronic health record for every patient in the data set. Examples of tuples are {“note:sepsis”, 1, 1000 seconds} and {“heart_rate_beats_per_minute”, 120, 1 day}.

At step 58, in order to deal with the time series nature of the data, we binarize all features as predicates and so real valued features might be represented by a predicate such as heart_rate>120 beats per minute within the last hour. The term “predicate” in this document is defined as a binary function which operates on a sequence of one or more of the tuples of step 56, or logical operations on sequences of the tuples. All predicates are functions that return 1 if true, 0 otherwise. As an example, a predicate Exists “heart_rate_beats_per_minute” in [{“heart_rate_beats_per_minute”, 120, 1 week} ] returns 1 because there is a tuple having {“heart “heart_rate_beats_per_minute”, 120, 1 day} in the entire sequence of heart_rate_beats_per_minute tuples over the sequence of the last week.

Predicates could also be logical combinations of binary functions on sequences of tuples, such as Exists Predicate 1 OR Predicate 2; or Exists Predicate 1 OR Predicate 2 where Predicate 2=(Predicate 2A AND Predicate 2B). As another example, a predicate could be combination of two Exists predicates for medications vancomycin AND zosyn over some time period.

At step 58, there is the optional step of grouping the predicates into two groups based on human understandability (i.e., understandable to an expert in the field). Examples of predicates in Group 1, which are the maximally human understandable predicates, are:
Exists: X—did the token/feature X exist at any point in a patient's timeline. Here X can be a word in a note, or the name of a lab or a procedure code among other things.
Counts: #X>C. Did the number of existences of the token/feature X over all time exceed C. More generally, a Counts predicate returns a result of 0 or 1 depending on the number of counts of a feature in the electronic health record data for a given patient relative to a numeric parameter C.

Depending on the type of prediction/label assigned by the model, other types of human understandable predicates could be selected as belonging to Group 1. Additionally, human understandable predicates could be generated or defined during model training by an operator or expert.

The predicates in Group 2, which are less human-understandable, can be for example:
Any x(ᵢ)>V at t(ᵢ)<T. Did the value of x(ᵢ) exceed V at time less than T in the past (or alternatively X<=V).
Max/Min/Avg_i x(ᵢ)>V. Did the maximum or minimum or average of X>V (or alternatively X<=V) over all time.
Hawkes process. Did the sum of exponential time decayed impulses when x(ᵢ)>V exceed some activation A over some time window T? Activation=sum_ᵢI(x(ᵢ)>V)*exp(−t(ᵢ)/T)
Decision List predicates where any two conjunctions of the above predicates are used.

In step 102 of FIG. 3, to build an interpretable model for generating fuzzy labels it may be desirable to only use human-understandable predicates as the features to use for the procedure of FIGS. 1 and 2. Once the training data has been labeled in accordance with procedure 102 of FIG. 1, it is then possible to build additional predictive models on top of the labelled training data. Examples of such predictive models are known in the technical and patent literature. For further details on several different types of predictive models and an ensemble of models for making health predications, see U.S. provisional patent application Ser. No. 62/538,112 filed Jul. 28, 2017 the content of which is incorporated by reference herein.

### Example

This example will describe an example of generation of a label of “dialysis” on an input training set of electronic health records. In this example, the training set was a small sample of 434 patients in the MIMIC-Ill data set, described above.

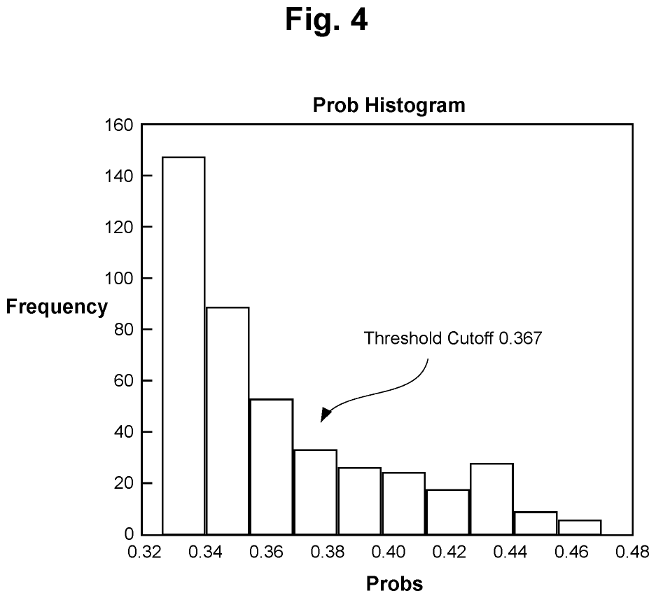

A. Buildup of partition predicates (steps 12, 14, 16, 18 of FIG. 1)
First Iteration through loop 18:
1. Seed (step 12): predicate selected by operator was “E:Composition.section.text.div.tokenized dialysis” (coverage: 32 examples, which is the number of examples triggered by this predicate.)
2. Steps 14 and 16: Machine provided a list and human chose:
   E:Composition.section.text.div.tokenized dialysis (coy: 32)
   E:Composition.section.text.div.tokenized esrd (coy: 17)
   #:Composition.section.text.div.tokenized dialysis>=2 (coy: 25)
   E:Composition.section.text.div.tokenized renal_failure (cov: 79)
      Second Iteration through loop 18:
1. Seed: 4 predicates from the first iteration.
2. Machine provided a list of predicates and human chose these additional ones:
   a. E: densePercentileTokens loinc_2160-0_0.9_mg_dl (coy: 35)
   b. E: densePercentileTokens loinc_2160-0_0.85_mg_dl (coy: 45)
   c. E: densePercentileTokens loinc_3094-0_0.8_mg_dl (cov: 71)
   (The predicates for feature type “densePercentileTokens” refer to laboratory tests (identified by the loinc” codes) and the test results are very high compared to the general population, which indicates illness. For example “E:densePercentileTokens loinc_2160-0_0.9_mg_dl” indicates that the patient had a Creatinine (loinc code 2160-0) measurement, measured in mg/dl, in the 90th percentile.)
   Third Iteration through loop 18:
1. Seed: 7 predicates from the second iteration.
2. Machine provided a list and human chose these additional ones:
   a. E: densePercentileTokens loinc_3094-0_0.85_mg_dl (coy: 61)
   b. E: densePercentileTokens loinc_2777-1_0.9_mg_dl (coy: 75)
   B. Second stage (steps 20, 22, 24 and 26 of FIG. 1)
1. We labeled the training data using the final predicate list: 9 predicates from the first stage. (step 20)
2. Build a model initialized with the 9 predicates. Allow the model to pick more predicates. (step 22)
3. Score the examples in the training set (step 24). Choose best threshold-cutoff (Prob=0.367) to get best F1 (=0.869). The performance metric area under the curve of a receiver operating characteristic plot (AUC/ROC) is 0.971 (independent of the threshold). Histogram is shown in FIG. 4.
4. Label the borderline examples (step 26). There are 59 examples within 1% of the probability threshold, i.e. 36.7%+/−1%. This represents 14% of the training data set. Depending on the human-evaluation resources we have, we can decide how many examples we can evaluate. This step is similar to “active learning” techniques in machine learning. Simple tools on a user interface of a computer can be used to present the patients within 1% of the probability threshold for human evaluation and assignment of labels to these samples.

Note that due to the shape of the distribution, this procedure will pick up more examples on the negative side. In this particular situation this is desirable, i.e. since the danger of false negative is greater, we want to inspect more examples on the negative side.

A validation of the above procedure can be performed as a “sanity check.” For example, we can evaluate the final model on a separate validation data set (1135 examples), against a related label “ccs:50” which is “Diabetes w/complications.” In this exercise we obtained a AUC/ROC=0.79 which is quite reasonable. This validates our approach. As a comparison, a model built based on the “ccs:50” label on the same training data set resulted in an AUC/ROC=0.88 on the same evaluation data set. The top predicates were medications related to the “ccs:50”, but nothing specific for dialysis.

A more formal evaluation in the form of a human evaluation on a small, uniformly sampled subset of the dataset is also possible. One could compute the accuracy of the fuzzy labels against those human-assigned labels.

### Further Considerations for the Example

It may be preferable to select features or predicates for fuzzy labelling which are “objective”, such as laboratory results, procedures, or medications. On the other hand, physician's notes are usually available only after the fact, and hence not very useful in a real-time clinical decision setting. It may be desirable to only use note terms when they are very specific to the labelling task.

In step 16 (and optionally in step 22 of FIG. 2) the coverage of each predicate is shown to the human expert. It turned out in this Example that the machine suggested predicates all have reasonable coverage, which is not surprising.

Some visualization of the suggested predicates in step 16 and optionally in step 22 may be useful, such as plotting their semantic relationships in a 2D plot with dot-size proportional to their weights.

Different human experts (104, FIG. 1) may result in different final list of predicates. One may want to investigate and evaluate the models compare with each other based on the different final lists of predicates. One metric can be based on “label flipping” in the human evaluation of labels at step 26. Less flipping means the human expert is more accurate.

In the second stage of the model generation (FIG. 1), including steps 20, 22, 24 and 26, there are several options on how this method could be executed, including:

1) Training the further boosting model of step 22 based on the labels from the current feature list creating a partition, as explained in detail above.
2) Allowing the domain expert (human-in-the-loop 104) to combine the predicates in a rule of their choice (e.g. “must have at least two of the following tokens present in notes “dialysis”, “esrd”, “renal_failure” and must have one of the following lab values: “E:densePercentileTokens loinc_3094-0_0.8_mg_dl”, “E:densePercentileTokens loinc_2160-0_0.85_mg_dl”, “E: densePercentileTokens loinc_2160-0_0.9_mg_dl (coy: 35)”).
3) Use active learning to strategically select unlabeled examples for human expert to evaluate, based on a certain policy.

As noted above, we may perform the second stage of the method of FIG. 1 several times to further refine the manually-assigned labels (step 26) using active learning.

1. A computer-implemented method of generating a class label for members of a set of training data, wherein the training data for each member in the set comprises a multitude of features, the method comprising the steps of executing the following instructions in a processor for the computer:
a) receiving an initial list of partition features from an operator which are conceptually related to the class label;
b) using the partition features to label the members of the set of training data and identifying additional partition features related to the class label which are not in the initial list of partition features with a boosting model;
c) adding selected ones of the additional partition features to the initial list of partition features from input by the operator;
d) repeating steps b) and c) one or more times to result in a final list of partition features;
e) using the final list of partition features from step d) to label the members of the set of training data;
f) building a further boosting model using the labels generated in step e);
g) scoring the members of the set of training data with the further boosting model of step f) and
h) generating labels from a subset of the members of the set of training data based on the scoring of step g) with input from the operator.

2. The method of claim 1, wherein step f) comprises the steps of initializing the further boosting model with the final list of partition features and iteratively generating additional features building a new further boosting model in each iteration.

3. The method of claim 1, wherein the iterations of generating additional features includes receiving operator input to deselect some of the generated additional features.

4. The method of claim 1, wherein the scoring step g) comprises determining a threshold related to the score, and identifying members of the set of training data within a range of the threshold, and wherein step h) comprises the step of generating labels for the identified members of the set of training data.

5. The method of claim 1, further comprising the step of building a predictive model from the set of training data with the labels assigned per steps e) and h).

6. The method of claim 1, wherein the members of the set of training data comprises a set of respective electronic health records.

7. The method of claim 6, wherein at least some of the features of the training data are associated with real values and a time component and such features in a tuple format of the type {X, xᵢ, tᵢ} where X is the name of feature, xᵢ is a real value of the feature and tᵢ is a time component for the real value xᵢ; and wherein the features comprise predicates defined as binary functions operating on sequences of the tuples or logical operations on the sequences of the tuples.

8. The method of claim 6, wherein at least some of the features of the training data are words contained in the health records, and at least some of the partition features are determinations of whether one or more words are present in the health records.

9. The method of claim 6, wherein at least some of the features in the training data are measurements in the health records, and at least some of the partition features are determinations of whether one or more measurements are present in the health records.

10. A computer-implemented method of generating a list of partition features for use in assigning a class label to members of a set of training data, wherein the training data for each member in the set comprises a multitude of features, the method comprising the steps of executing the following instructions in a processor for the computer:
a) receiving an initial list of partition features from an operator which are conceptually related to the class label;
b) using the initial list of partition features to label the members of the set of training data and identify additional partition features related to the class label which are not in the initial list of partition features with a boosting model;
c) adding selected ones of the additional partition features to the initial list of partition features from input by the operator to result in an updated list of partition features;
d) repeating steps b) and c) one or more times using the updated list of partition features as the input in step b) to result in a final list of partition features.

11. The method of claim 10, wherein step b) comprises iteratively building a boosting model initialized with the initial set of partition features, and wherein in each iteration of building the boosting model additional partition features are identified.

12. The method of claim 10, wherein the method further comprises a step of using the final list of partition features to generate the class label for members of the set of training data.

13. The method of claim 10, wherein the set of training data comprises a set of electronic health records.

14. The method of claim 13, wherein training data is associated with real values and a time component and is in a tuple format of the type {X, xᵢ, tᵢ} where X is the name of feature, xᵢ is a real value of the feature and tᵢ is a time component for the real value xᵢ; and the features comprise predicates defined as binary functions operating on sequences of the tuples or logical operations on sequences of the tuples.

15. A computer-implemented method for generating a class label for members of a set of training data, wherein the training data for each member in the set comprises a multitude of features, the method comprising the steps of the executing the following instructions in a processor for the computer:
(a) using a first boosting model with user input from to gradually build up a list of partition features;
(b) labeling the members of the set of training data with the list of partition features;
(c) building a further boosting model from the labeled members of the set of training data and generating additional partition features;
(d) scoring the labeling of the members of the set of training data and determining a threshold;
(e) identifying a subset of members of the set of training data within a range of the threshold; and
(f) assigning labels to the subset of members with user input.

16. The method of claim 15, wherein the set of training data comprises a set of electronic health records.

17. The method of claim 16, wherein training data is associated with real values and a time component and is in a tuple format of the type {X, xᵢ, tᵢ} where X is the name of feature, xᵢ is a real value of the feature and tᵢ is a time component for the real value xᵢ; and the features comprise predicates defined as binary functions operating on sequences of the tuples or logical operations on sequences of the tuples.

18. The method of claim 15, further comprising the steps of repeating steps (c), (d), (e) and (f) at least one time.

19. The method of claim 16, further comprising the step of building a predictive model from the set of training data with the labels assigned per steps b) and f).

20. A computer-implemented method of generating a respective class label for each of a plurality of members of a set of training data, wherein the training data for each member in the set comprises a multitude of features, the method comprising the steps of executing the following instructions in a processor for the computer:
a) receiving an initial list of tests from an operator which are conceptually related to the class label, each test being a function applicable to any member of the training data to detect one or more of the features in that member of the training data;
b) using the test to label the training data and identify additional tests related to the class label which are not in the initial list of tests;
c) adding selected ones of the additional tests to the initial list of tests based on data from input by the operator;
d) repeating steps b) and c) one or more times to result in a final list of tests;
e) using the final list of tests from step d) to label the training data;
f) building a boosting model using the labels generated in step e);
g) scoring the training examples with the boosting model built in step f) and
h) generating respective labels for a subset of the members of the training examples based on the scoring of step g) with input from the operator.

21. The method of claim 20, wherein in step b) the additional tests are generated using a boosting model.

22. The method of claim 20, wherein step f) comprises the steps of initializing the further boosting model with the final list of tests and iteratively generating additional tests building a new boosting model in each iteration.

23. The method of claim 22 wherein the iterations of generating additional tests include receiving operator input to deselect some of the generated additional tests.

24. The method of claim 20, wherein the scoring step g) comprises determining a threshold related to the score, and identifying members of the training data for which the score differs from the threshold by an amount within a pre-defined range, and wherein step h) comprises the step of generating labels for the identified members of the set of training data.

25. The method of claim 20, further comprising the step of building a predictive model from the set of samples with the labels assigned per steps e) and h).

26. The method of claim 20, wherein the members of the set of training data comprises a set of respective electronic health records.

27. The method of claim 26, wherein at least some features of the training data are associated with real values and a time component and is in a tuple format of the type {X, xᵢ, tᵢ} where X is a name of feature, xᵢ is a real value of the feature and tᵢ is a time component for the real value xᵢ; and the tests comprise predicates defined as binary functions operating on sequences of the tuples or logical operations on the sequences of the tuples.

28. The method of claim 26, wherein at least some features of the training data are words contained in the health records, and at least some of the tests are determinations of whether one of more corresponding predetermined words are present in the health records.

29. The method of claim 26, wherein at least some of the features in the training data are measurements in the health records, and at least some of the features are determinations of whether one or more measurements are present in the health records.
